(19) United States
(12) Patent Application Publication
(10) Pub. No.: US 2013/0165417 A1
(43) Pub. Date: Jun. 27, 2013

(54) METHODS AND COMPOSITIONS FOR TREATING ACE2-RELATED DISORDERS

(71) Applicants: Mohan K. Raizada, Alachua, FL (US); David A. Ostrov, Gainesville, FL (US); Michael J. Katovich, Gainesville, FL (US)

(72) Inventors: Mohan K. Raizada, Alachua, FL (US); David A. Ostrov, Gainesville, FL (US); Michael J. Katovich, Gainesville, FL (US)

(73) Assignee: UNIVERSITY OF FLORIDA RESEARCH FOUNDATION, Gainesville, FL (US)

(21) Appl. No.: 13/643,080

(22) PCT Filed: Apr. 23, 2011
(86) PCT No.: PCT/US11/33707

§ 371 Date: Mar. 6, 2013

Related U.S. Application Data
(60) Provisional application No. 61/327,651, filed on Apr. 23, 2010.

(51) Int. Cl. A61K 31/655

(52) U.S. Cl. CPC A61K 31/655
USPC 514/151

(57) ABSTRACT

The invention relates to methods of treating cardiovascular and cardiopulmonary diseases and associated conditions, including hypertension. The invention further relates to pharmaceutical compositions for treating cardiovascular and cardiopulmonary diseases, especially hypertension, and lung injury.

### RELATED APPLICATIONS

This application claims the benefit of and priority to U.S. Provisional Patent Application No. 61/327,651, filed Apr. 23, 2010, the contents of which are incorporated herein by reference in their entirety.

### STATEMENT OF RIGHTS TO INVENTIONS MADE UNDER FEDERALLY SPONSORED RESEARCH

This work was supported by the National Institutes of Health, Grant No. HL56921. The government has certain rights in the invention.

### BACKGROUND OF THE INVENTION

ACE2 is a family member of the peptidylpeptidase angiotensin-converting enzymes (ACE), which are reviewed in Kem & Brown, N. Eng. J. Med. 323(16) 1136-1137 (1990), see also Yamada et al, Circ. Res. 68 141-149 (1991). There are three ACE enzymes currently known, ACE1, ACE2 and ACE3. (Cambien et al, Am. J. Hum. Genet. 43 774-780 (1988); Mattu et al. Circulation 91 270-274 (1995); Rigat et al, Nuc. Acids. Res. 20(6) 1433 (1992)). The human ACE gene (DCP1) is found on chromosome 17q23 and contains a restriction fragment length polymorphism consisting of the presence (Insertion, I) or absence (Deletion, D) of a 287 base pair alu repeat sequence in intron 16. ACE-2 (GenBank Accession No. AF291820) has been described by Donoghue, et al. (2000) Circ. Res. 87:e1-e9. ACE2 cleaves angiotensin I, but ACE-2 is a carboxypeptidase. The nucleic acid and amino acid sequences of ACE-2 reveal that certain portions of the ACE-2 protein and cDNA have a significant homology to certain regions of previously identified angiotensin converting enzymes (Altschul et al. J. Mol. Biol. (1990) 215:403).

The crystal structure of ACE2 was solved and revealed a “hinge” that is inhibitor-dependent and brings catalytic residues into position. Towler P, Staker B, Prasad S G, Menon S, Tang J, Parsons T, Ryan D, Fisher M, Williams D, Dales N A, Patane M A, and Pantoliano M W, ACE2 X-ray structures reveal a large hinge-bending motion important for inhibitor binding and catalysis, J Biol. Chem. 2004, 23; 279(17):17996-8007.

Angiotensin-converting enzyme 2 (ACE2) is a type I membrane-anchored peptidyl carboxypeptidase of 805 amino acids (Donoghue et al. 2000, Tipnis et al. 2000). Its catalytic domain consists of approximately 733 residues and is 42% identical to that of its closest homolog, ACE. Unlike the ubiquitously expressed ACE, ACE2 is expressed only in the kidneys, heart (including all cardiovascular tissues), and lungs (Donoghue et al. 2000). Its substrate specificity has also been established to be different, and likely complementary, to that of ACE (Vickers et al. 2002). While ACE activity mainly results in the production of angiotensin II involved in vasoconstriction and the biosynthesis of aldosterone (an important regulator of blood pressure), ACE2 product peptides, namely angiotensin 1-7, are involved in vasodilation and hypotension. Furthermore, inhibitors of ACE such as captopril, lisinopril and enalaprilat do not significantly affect the activity of ACE2 (Donoghue et al. 2000, Tipnis et al. 2000).

Specific roles of ACE2 in different diseases and normal physiology are currently a subject of intense study. Nonetheless, its central role in the renin-angiotensin system (Burrel et al. 2004), cardiac contractile function (Crackower et al. 2002), hypertension (Katovich et al. 2005) and therefore cardiovascular disease have all been recently established. Crackower and others (2002) also observed an inverse correlation of ACE2 mRNA and blood pressure in experimental hypertension models. Other studies have begun to demonstrate ACE2 represents a tractable gene therapy target (Katovich et al. 2005; Huentelman et al. 2004). The approach attempts to over-express ACE2 to offer protection against cardiac hypertrophy and fibrosis (Katovich et al. 2005). The inhibition of ACE is an established therapeutic approach and presently one of the primary strategies for the treatment of hypertension. However these studies (mentioned above) clearly suggests that suppression of ACE and enhancement of ACE2 activity are both highly desirable to prevent and treat hypertension and related cardiovascular diseases.

### BRIEF SUMMARY OF THE INVENTION

In one aspect, the invention provides a method of treating a subject suffering from or susceptible to cardiovascular disease, the method comprising administering to a subject in need thereof a therapeutically effective amount of diminazene or a pharmaceutically acceptable salt thereof, to thereby treat the subject suffering from or susceptible to cardiovascular disease.

In another aspect, the invention provides a method of treating a subject suffering from or susceptible to hypertension disease, the method comprising administering to a subject in need thereof a therapeutically effective amount of diminazene or a pharmaceutically acceptable salt thereof, to thereby treat the subject suffering from or susceptible to hypertension.

In certain embodiments, the diminazene or a pharmaceutically acceptable salt is diminazene aceturate.

In another aspect, the invention provides a kit for treating cardiovascular disease or an associated condition in a subject, comprising diminazene or a pharmaceutically acceptable salt thereof, and instructions for use.

In another aspect, the invention provides a method of treating a subject suffering from or susceptible to acute lung injury, comprising administering to the subject an effective amount of diminazene or a pharmaceutically acceptable salt thereof, such that the subject is treated.

In another aspect, the invention provides a method of treating a subject suffering from or susceptible to liver, cardiac, pulmonary, vascular or renal fibrosis, the method comprising administering to the subject an effective amount of diminazene or a pharmaceutically acceptable salt thereof, such that the subject is treated.

In another aspect, the invention provides a method of treating a subject suffering from or susceptible to pulmonary hypertension, the method comprising administering to the subject an effective amount of diminazene or a pharmaceutically acceptable salt thereof, such that the subject is treated.

In another aspect, the invention provides a method of treating a subject suffering from or susceptible to chronic obstructive pulmonary disease (COPD), the method comprising administering to the subject an effective amount of diminazene or a pharmaceutically acceptable salt thereof, such that the subject is treated.

In another aspect, the invention provides a method of treating a subject suffering from or susceptible to heart failure, the method comprising administering to the subject an effective amount of diminazene or a pharmaceutically acceptable salt thereof, such that the subject is treated.

In another aspect, the invention provides a method of treating a subject suffering from or susceptible to diabetes or a diabetes-associated condition, the method comprising administering to the subject an effective amount of diminazene or a pharmaceutically acceptable salt thereof, such that the subject is treated.

In another aspect, the invention provides a method of treating a subject suffering from or susceptible to cardiac damage induced by anticancer drugs, the method comprising administering to the subject an effective amount of diminazene or a pharmaceutically acceptable salt thereof, such that the subject is treated.

In another aspect, the invention provides a method of treating a subject suffering from or susceptible to cardiac damage from myocardial infarction, the method comprising administering to the subject an effective amount of diminazene or a pharmaceutically acceptable salt thereof, such that the cardiac damage from myocardial infarction is treated or prevented.

In another aspect, the invention provides a method of treating or preventing progression of tissue damage or pathophysiology in a subject suffering from pulmonary hypertension, the method comprising administering to the subject suffering from pulmonary hypertension an effective amount of diminazene or a pharmaceutically acceptable salt thereof, such that the progression of tissue damage or pathophysiology is treated or prevented.

In another aspect, the invention provides a method of improving cardiac function in a subject suffering from myocardial infarction, the method comprising administering to the subject an effective amount of diminazene or a pharmaceutically acceptable salt thereof, such that cardiac function in the subject is improved.

In another aspect, the invention provides a method of treating a subject suffering from or susceptible to liver damage, the method comprising administering to the subject an effective amount of diminazene or a pharmaceutically acceptable salt thereof, such that the subject is treated.

In another aspect, the invention provides a method of treating a subject suffering from or susceptible to stroke, the method comprising administering to the subject an effective amount of diminazene or a pharmaceutically acceptable salt thereof, such that the subject is treated.

In any of the methods or kits of the invention, the diminazene or a pharmaceutically acceptable salt thereof can be diminazene aceturate.

In another aspect, the invention provides a pharmaceutical composition comprising diminazene or a pharmaceutically acceptable salt thereof and a pharmaceutically acceptable carrier, in an oral dosage form. In one embodiment, the diminazene or a pharmaceutically acceptable salt thereof is diminazene aceturate.

Other aspects and embodiments of the invention are disclosed infra.

### BRIEF DESCRIPTION OF THE DRAWINGS

The present invention is further described below with reference to the following non-limiting examples and with reference to the following figures, in which:

FIG. 1 depicts DIZE (diminazene aceturate, also known as 4,4′-(1-Triazene-1,3-diyl)bis(benzenecarboximidamide) or 4-[2-(4-carbamimidoylphenyl)iminohydrazinyl]benzenecarboximidamide) and XNT (1-[[2-(dimethylamino)ethyl]amino]-4-(hydroxymethyl)-7-[[4-methylphenylsulfonyl]oxy]-9H-xanthenone) docked onto Site 1 of ACE2.

FIG. 2 is a graph showing that DIZE increases ACE2 but not ACE activity.

FIG. 3 is a graph showing that DIZE attenuates myocardial infarction-induced (MI-induced) cardiac dysfunctions.

FIG. 4 is a pair of graphs showing that DIZE prevents increase in Right Ventricular Systolic Pressure (RVSP) and Right Ventricular Hypertrophy (RVH) in monocrotaline (MCT) treated rats.

FIG. 5 is a pair of graphs showing that DIZE reverses increases in RVSP and RVH in MCT-treated rats.

FIG. 6 shows that DIZE treatment causes reversal of MCT-induced right ventricular fibrosis.

FIG. 7. Identification of diminazene aceturate (DIZE) as an ACE2 activator. A. Cartoon representation of the three identified sites present in the ACE2 enzyme: the hinge, backside and the lip. B. Schematic representation of the structure of XNT, DIZE and of both compounds occupying the predicted binding site. C. DIZE enhances specifically the ACE2 enzyme activity.

FIG. 8. A dose response decrease in blood pressure following the administration of DIZE. WKY rats (upper panel) and SHRs (lower panel) were injected with an intravenous bolus containing either vehicle (sterile saline 0.9%) or increasing doses of DIZE as indicated in the graph. The asterix denotes statistical significance *p<0.01, **p<0.001, ***p<0.0001 compared to vehicle control, N=6.

FIG. 9. Heart rate of WKY and SHR animals upon DIZEs acute administration. Heart rate data were collected upon DIZEs administration as IV bolus at the indicated doses. Upper panel WKY, N=5; lower panel SHR, N=6.

### DETAILED DESCRIPTION OF THE INVENTION

### 1. Definitions

Before further description of the present invention, and in order that the invention may be more readily understood, certain terms are first defined and collected here for convenience.

As used herein, the term “acute lung injury” refers to conditions generally involving bilateral pulmonary infiltrates on chest X-ray, a pulmonary capillary wedge pressure of less than 18 mm Hg, and a PaO₂/FiO₂ of less than 300. Acute lung injury includes hypoxemic respiratory syndrome and acute respiratory distress syndrome (ARDS). ARDS is one of the most severe forms of acute lung injury. ARDS is a serious clinical syndrome with a high mortality rate (30-60%). ARDS may be caused by include sepsis, pulmonary aspiration, pneumonias, major trauma, burns, and infections (e.g., with the severe acute respiratory syndrome (SARS) coronavirus).

The term “administration” or “administering” includes routes of introducing the compound of the invention(s) to a subject to perform their intended function. Examples of routes of administration that may be used include injection (subcutaneous, intravenous, parenterally, intraperitoneally, intrathecal), oral, inhalation, rectal and transdermal. The pharmaceutical preparations may be given by forms suitable for each administration route. For example, these preparations are administered in tablets or capsule form, by injection, inhalation, eye lotion, ointment, suppository, etc. administration by injection, infusion or inhalation; topical by lotion or ointment; and rectal by suppositories. Oral administration is preferred. The injection can be bolus or can be continuous infusion. Depending on the route of administration, the compound of the invention can be coated with or disposed in a selected material to protect it from natural conditions which may detrimentally effect its ability to perform its intended function. The compound of the invention can be administered alone, or in conjunction with either another agent as described above or with a pharmaceutically-acceptable carrier, or both. The compound of the invention can be administered prior to the administration of the other agent, simultaneously with the agent, or after the administration of the agent. Furthermore, the compound of the invention can also be administered in a proform which is converted into its active metabolite, or more active metabolite in vivo.

The language “biological activities” of a compound of the invention includes all activities elicited by compound of the inventions in a responsive cell or subject. It includes genomic and non-genomic activities elicited by these compounds.

“Biological composition” or “biological sample” refers to a composition containing or derived from cells or biopolymers. Cell-containing compositions include, for example, mammalian blood, red cell concentrates, platelet concentrates, leukocyte concentrates, blood cell proteins, blood plasma, platelet-rich plasma, a plasma concentrate, a precipitate from any fractionation of the plasma, a supernatant from any fractionation of the plasma, blood plasma protein fractions, purified or partially purified blood proteins or other components, serum, semen, mammalian colostrum, milk, saliva, placental extracts, a cryoprecipitate, a cryosupernatant, a cell lysate, mammalian cell culture or culture medium, products of fermentation, ascites fluid, proteins induced in blood cells, and products produced in cell culture by normal or transformed cells (e.g., via recombinant DNA or monoclonal antibody technology). Biological compositions can be cell-free. In a preferred embodiment, a suitable biological composition or biological sample is a red blood cell suspension. In some embodiments, the blood cell suspension includes mammalian blood cells. Preferably, the blood cells are obtained from a human, a non-human primate, a dog, a cat, a horse, a cow, a goat, a sheep or a pig. In preferred embodiments, the blood cell suspension includes red blood cells and/or platelets and/or leukocytes and/or bone marrow cells.

The term “chiral” refers to molecules which have the property of non-superimposability of the minor image partner, while the term “achiral” refers to molecules which are superimposable on their minor image partner.

The term “diastereomers” refers to stereoisomers with two or more centers of dissymmetry and whose molecules are not minor images of one another.

The term “effective amount” includes an amount effective, at dosages and for periods of time necessary, to achieve the desired result, e.g., sufficient to treat cardiovascular disease or an associated condition. An effective amount of compound of the invention may vary according to factors such as the disease state, age, and weight of the subject, and the ability of the compound of the invention to elicit a desired response in the subject. Dosage regimens may be adjusted to provide the optimum therapeutic response. An effective amount is also one in which any toxic or detrimental effects (e.g., side effects) of the compound of the invention are outweighed by the therapeutically beneficial effects.

A therapeutically effective amount of compound of the invention (i.e., an effective dosage) may range from about 0.001 to 30 mg/kg body weight, or from about 0.01 to 10 mg/kg body weight, or from about 0.05 to 5 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound of the invention can include a single treatment or, preferably, can include a series of treatments. In certain embodiments, a therapeutically effective amount of diminazene or a pharmaceutically acceptable salt thereof, e.g., diminazene aceturate, can be, e.g., subcutaneous administration of e.g., 1-5 mg/kg/day chronically, or oral administration of, e.g., 0.1-5 mg/kg/day chronically. It will also be appreciated that the effective dosage of a compound of the invention used for treatment may increase or decrease over the course of a particular treatment.

The term “enantiomers” refers to two stereoisomers of a compound which are non-superimposable mirror images of one another. An equimolar mixture of two enantiomers is called a “racemic mixture” or a “racemate.”

The term “homeostasis” is art-recognized to mean maintenance of static, or constant, conditions in an internal environment.

The language “improved biological properties” refers to any activity inherent in a compound of the invention that enhances its effectiveness in vivo. In a preferred embodiment, this term refers to any qualitative or quantitative improved therapeutic property of a compound of the invention, such as reduced toxicity.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The term “prodrug” includes compounds with moieties that can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included.

The language “a prophylactically effective amount” of a compound refers to diminazene or a pharmaceutically acceptable salt (including diminazene aceturate) which is effective, upon single or multiple dose administration to the patient, in preventing or treating cardiovascular disease or cardiopulmonary disease or hypertension or cardiac or renal fibrosis, or another condition as described herein.

The language “reduced toxicity” is intended to include a reduction in any undesired side effect elicited by a compound of the invention when administered in vivo.

The term “subject” includes organisms which are capable of suffering from cardiovascular disease, or an associated condition (including hypertension) or who could otherwise benefit from the administration of a compound of the invention of the invention, such as human and non-human animals. Preferred human animals include human patients suffering from or prone to suffering from cardiovascular disease or associated state, including hypertension, or another condition treatable by administration of diminazene or a pharmaceutically acceptable salt as described herein. The term “non-human animals” of the invention includes all vertebrates, e.g., mammals, e.g., rodents, e.g., mice, and non-mammals, such as non-human primates, e.g., sheep, dog, cow, chickens, amphibians, reptiles, etc. “Susceptible to a cardiovascular disease or associated state, including hypertension” is meant to include subjects at risk of developing cardiovascular disease or associated state, including hypertension, i.e., subjects suffering from existing cardiovascular disease or associated state, including hypertension, subjects having risk factors (such as overweight) for cardiovascular disease or associated state, including hypertension, etc.

The phrases “systemic administration,” “administered systemically”, “peripheral administration” and “administered peripherally” as used herein mean the administration of a compound of the invention(s), drug or other material, such that it enters the patient's system and, thus, is subject to metabolism and other like processes, for example, subcutaneous administration.

The language “therapeutically effective amount” of diminazene or a pharmaceutically acceptable salt thereof refers to an amount of an agent which is effective, upon single or multiple dose administration to the patient, in treating or preventing cardiovascular disease or an associated condition or symptom, including hypertension, or in prolonging the survivability of the patient with such condition beyond that expected in the absence of such treatment.

The language “cardiovascular disease or associated condition” refers to a condition of the heart or vasculature, including heart disease and stroke, which can be prevented, treated or otherwise ameliorated by administration of one or more compounds of the invention (e.g., is caused, exacerbated or characterized by insufficient ACE2 activity). Other examples of cardiovascular disease or associated conditions include cardiac hypertrophy and fibrosis.

The language “diabetes or a diabetes-associated condition” refers to diabetes (such as Type I diabetes, Type II diabetes, or gestational diabetes) and conditions related to or associated with diabetes, which can be prevented, treated or otherwise ameliorated by administration of one or more compounds of the invention (e.g., is caused, exacerbated or characterized by insufficient ACE2 activity). Examples of diabetes-associated conditions include cardiac, renal, and retinal damage, peripheral neuropathy, and the like.

With respect to the nomenclature of a chiral center, terms “d” and “1” configuration are as defined by the IUPAC Recommendations. As to the use of the terms, diastereomer, racemate, epimer and enantiomer will be used in their normal context to describe the stereochemistry of preparations.

### 2. Compounds of the Invention

In one aspect, the invention provides a compound, i.e., diminazene or a pharmaceutically acceptable salt thereof, capable of activating ACE2 activity. In certain embodiments, the compound is capable of activating or increasing ACE2 activity selectively, e.g., without concomitant activation of ACE activity. In certain embodiments, the ACE2 activator compound is diminazene aceturate (diminazene N-acetylglycinate).

In general, a compound of the invention will be selected such that the compound is capable of binding to a binding pocket of ACE2 that is defined (at least in part) by structure coordinates of one or more of ACE2 amino acid residues Lys94, Tyr196, Gly205 and His195, or is capable of binding to a binding pocket of ACE2 that is defined (at least in part) by structure coordinates of one or more of ACE2 amino acid residues Gln98, Gln101 and Gly205.

Diminazene (and diminazene aceturate) is a known compound and can be purchased from commercial sources or prepared according to techniques known in the art. Furthermore, compounds of the invention can be purified, separated, or isolated, e.g., by crystallization, chromatographic separation (e.g., by liquid chromatography), or by other methods known in the art.

As used herein, the term “pharmaceutically acceptable salt,” is a salt formed from an acid and diminazene. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, aceturate and pamoate (i.e., 1,1′-methylene-bis-(2-hydroxy-3-naphthoate)) salts.

### 3. Uses of the Compounds of the Invention

As described herein below, it has now surprisingly been found that the compounds of the invention and analogs can treat and prevent cardiovascular diseases, including systemic hypertension or pulmonary hypertension. Thus, in one embodiment, the invention provides a method of treating a subject suffering from or susceptible to cardiovascular disease or systemic or pulmonary hypertension comprising administering to subject in need thereof a therapeutically effective amount of a compound capable of activating ACE2, or a pharmaceutically acceptable salt or prodrug thereof. In one embodiment, the compound is capable of binding to or interacting with a binding pocket defined (at least in part) by structure coordinates of one or more ACE2 amino acid residues Lys94, Tyr196, Gly205 and His195. In another embodiment, the compound is capable of binding to or interacting with a binding pocket defined (at least in part) by structure coordinates of one or more ACE2 residues Gln98, Gln101 and Gly205. In certain embodiments, the diminazene or a pharmaceutically acceptable salt is diminazene aceturate.

In another aspect, the invention provides a method of treating a subject suffering from or susceptible to cardiovascular disease or hypertension, comprising administering to the subject an effective amount of a compound capable of activating ACE2 activity or expression in a cell, such that the subject is treated.

In one aspect, the invention provides a method of treating a subject suffering from or susceptible to cardiovascular disease or hypertension comprising administering to subject in need thereof diminazene or a pharmaceutically acceptable salt or prodrug thereof. In certain embodiments, the compound is diminazene aceturate.

In another aspect, the invention provides a kit for treating cardiovascular disease or an associated condition in a subject, comprising diminazene or a pharmaceutically acceptable salt thereof, and instructions for use.

In another aspect, the invention provides a method of treating a subject suffering from or susceptible to acute lung injury, comprising administering to the subject an effective amount of diminazene or a pharmaceutically acceptable salt thereof, such that the subject is treated.

In another aspect, the invention provides a method of treating a subject suffering from or susceptible to liver, cardiac, pulmonary, vascular or renal fibrosis, the method comprising administering to the subject an effective amount of diminazene or a pharmaceutically acceptable salt thereof, such that the subject is treated. In certain embodiments, a method of treating a subject suffering from liver, cardiac, pulmonary, vascular or renal fibrosis includes ameliorating, decreasing the extent of, or reversing liver, cardiac, pulmonary, vascular or renal fibrosis in an organ or a subject.

In another aspect, the invention provides a method of treating a subject suffering from or susceptible to pulmonary hypertension, the method comprising administering to the subject an effective amount of diminazene or a pharmaceutically acceptable salt thereof, such that the subject is treated.

In another aspect, the invention provides a method of treating a subject suffering from or susceptible to chronic obstructive pulmonary disease (COPD), the method comprising administering to the subject an effective amount of diminazene or a pharmaceutically acceptable salt thereof, such that the subject is treated.

In another aspect, the invention provides a method of treating a subject suffering from or susceptible to heart failure, the method comprising administering to the subject an effective amount of diminazene or a pharmaceutically acceptable salt thereof, such that the subject is treated.

In another aspect, the invention provides a method of treating a subject suffering from or susceptible to diabetes or a diabetes-associated condition, the method comprising administering to the subject an effective amount of diminazene or a pharmaceutically acceptable salt thereof, such that the subject is treated.

In another aspect, the invention provides a method of treating a subject suffering from or susceptible to cardiac damage induced by anticancer drugs, the method comprising administering to the subject an effective amount of diminazene or a pharmaceutically acceptable salt thereof, such that the subject is treated.

In another aspect, the invention provides a method of treating a subject suffering from or susceptible to cardiac damage from myocardial infarction, the method comprising administering to the subject an effective amount of diminazene or a pharmaceutically acceptable salt thereof, such that the subject is treated.

In another aspect, the invention provides a method of treating or preventing progression of tissue damage or pathophysiology in a subject suffering from pulmonary hypertension, the method comprising administering to the subject suffering from pulmonary hypertension an effective amount of diminazene or a pharmaceutically acceptable salt thereof, such that the subject is treated.

In another aspect, the invention provides a method of improving cardiac function in a subject suffering from myocardial infarction, the method comprising administering to the subject an effective amount of diminazene or a pharmaceutically acceptable salt thereof, such that the subject is treated.

In another aspect, the invention provides a method of treating a subject suffering from or susceptible to liver damage, the method comprising administering to the subject an effective amount of diminazene or a pharmaceutically acceptable salt thereof, such that the subject is treated.

In another aspect, the invention provides a method of treating a subject suffering from or susceptible to stroke, the method comprising administering to the subject an effective amount of diminazene or a pharmaceutically acceptable salt thereof, such that the subject is treated.

In certain aspects, the invention provides methods for treating or preventing a condition selected from the group consisting of: hypertension, including pulmonary hypertension; pulmonary fibrosis; COPD; cardiovascular diseases including heart failure, cardiac, renal and vascular fibrosis; diabetes and its associated effects on cardiac, renal and retinal damages; liver damage and fibrosis; cardiac damage induced by anticancer drugs.

In another aspect, the invention provides a method of improving function of endothelial progenitor cells in a pulmonary hypertensive subject, the method comprising administering to the subject an effective amount of diminazene or a pharmaceutically acceptable salt thereof, such that the function of endothelial progenitor cells in the subject is improved.

In any of the methods of the invention, the diminazene or a pharmaceutically acceptable salt thereof can be diminazene aceturate.

In another aspect, the invention provides a pharmaceutical composition comprising diminazene or a pharmaceutically acceptable salt thereof and a pharmaceutically acceptable carrier, in an oral dosage form. In one embodiment, the diminazene or a pharmaceutically acceptable salt thereof is diminazene aceturate.

In another aspect, the invention provides a method for increasing activity or expression of ACE2 in vitro, or in a cell or a subject, the method comprising contacting the cell or subject with an effective amount of diminazene or a pharmaceutically acceptable salt thereof, such that activity or expression of ACE2 is increased.

In certain embodiments, the methods of the invention include administering to a subject a therapeutically effective amount of diminazene or a pharmaceutically acceptable salt thereof in combination with another pharmaceutically active compound. Examples of pharmaceutically active compounds include compounds known to treat cardiovascular disease or hypertension, such as ACE inhibitors, angiotension II receptor blockers, diuretics, beta blockers, calcium channel blockers, statins, aspirin, and the like. Other pharmaceutically active compounds that may be used can be found in Harrison's Principles of Internal Medicine, Thirteenth Edition, Eds. T. R. Harrison et al. McGraw-Hill N.Y., NY; and the Physicians Desk Reference 50th Edition 1997, Oradell N.J., Medical Economics Co., the complete contents of which are expressly incorporated herein by reference. The compound of the invention and the pharmaceutically active compound may be administered to the subject in the same pharmaceutical composition or in different pharmaceutical compositions (at the same time or at different times).

Determination of a therapeutically effective amount or a prophylactically effective amount of the compound of the invention, can be readily made by the physician or veterinarian (the “attending clinician”), as one skilled in the art, by the use of known techniques and by observing results obtained under analogous circumstances. The dosages may be varied depending upon the requirements of the patient in the judgment of the attending clinician; the severity of the condition being treated and the particular compound being employed. In determining the therapeutically effective amount or dose, and the prophylactically effective amount or dose, a number of factors are considered by the attending clinician, including, but not limited to: the specific cardiovascular disease or condition involved; pharmacodynamic characteristics of the particular agent and its mode and route of administration; the desired time course of treatment; the species of mammal; its size, age, and general health; the degree of or involvement or the severity of the disease; the response of the individual patient; the particular compound administered; the mode of administration; the bioavailability characteristics of the preparation administered; the dose regimen selected; the kind of concurrent treatment (i.e., the interaction of the compound of the invention with other co-administered therapeutics); and other relevant circumstances.

Treatment can be initiated with smaller dosages, which are less than the optimum dose of the compound. Thereafter, the dosage may be increased by small increments until the optimum effect under the circumstances is reached. For convenience, the total daily dosage may be divided and administered in portions during the day if desired. A therapeutically effective amount and a prophylactically effective amount of a compound of the invention of the invention is expected to vary from about 0.1 milligram per kilogram of body weight per day (mg/kg/day) to about 100 mg/kg/day.

Compounds determined to be effective for the prevention or treatment of cardiovascular disease in animals, e.g., dogs, chickens, and rodents, may also be useful in treatment of similar conditions in humans. Those skilled in the art of treatment in humans will know, based upon the data obtained in animal studies, the dosage and route of administration of the compound to humans. In general, the dosage and route of administration in humans is expected to be similar to that in animals.

The identification of those patients who are in need of prophylactic treatment for cardiovascular disease states, pulmonary hypertension, fibrosis, or other conditions as described herein is well within the ability and knowledge of one skilled in the art. Certain of the methods for identification of patients which are at risk of developing cardiovascular disease states which can be treated by the subject methods are appreciated in the medical arts, such as family history, the presence of other risk factors associated with the development of that disease state in the subject patient, and the like. A clinician skilled in the art can readily identify such candidate patients, by the use of, for example, clinical tests, physical examination and medical/family/travel history.

A method of assessing the efficacy of an anti-cardiovascular disease treatment in a subject includes determining the physical condition of the subject (e.g., blood pressure, degree or extent of atherosclerosis, and the like) and then administering a therapeutically effective amount of an ACE activator compound of the invention to the subject. After a appropriate period of time after the administration of the compound, e.g., 2 hours, 4 hours, 8 hours, 12 hours, or 72 hours, or one week, the physical condition of the subject is determined again. The modulation of the cardiovascular disease state indicates efficacy of an treatment. The physical condition of the subject may be determined periodically throughout treatment. For example, the physical condition of the subject may be checked every few hours, days or weeks to assess the further efficacy of the treatment. The method described may be used to screen or select patients that may benefit from treatment with an ACE activator.

As used herein, “obtaining a biological sample from a subject,” includes obtaining a sample for use in the methods described herein. A biological sample is described above.

In another aspect, a compound of the invention is packaged in a therapeutically effective amount with a pharmaceutically acceptable carrier or diluent. The composition may be formulated for treating a subject suffering from or susceptible to a cardiovascular disease or associated condition, and packaged with instructions to treat a subject suffering from or susceptible to such a disease or condition.

In another aspect, the invention provides a method for increasing activity or expression of ACE2 in a cell or a subject, the method comprising contacting the cell or subject with an effective amount of diminazene or a pharmaceutically acceptable salt thereof, such that activity or expression of ACE2 is increased.

In another aspect, the invention provides a packaged composition including a therapeutically effective amount of diminazene or a pharmaceutically acceptable salt thereof and a pharmaceutically acceptable carrier or diluent. The composition may be formulated for treating a subject suffering from or susceptible to cardiovascular disease or an associated condition (such as stroke or heart disease), or hypertension, or fibrosis and packaged with instructions to treat a subject suffering from or susceptible to cardiovascular disease or an associated condition (such as stroke or heart disease), or hypertension.

In one aspect, the invention provides a kit for treating cardiovascular disease or an associated condition (such as stroke or heart disease), or hypertension, in a subject is provided and includes a compound disclosed herein, e.g., diminazene or a pharmaceutically acceptable salt or prodrug thereof, and instructions for use. In further aspects, the invention provides kits for treating cardiovascular disease or an associated condition (such as stroke or heart disease), or hypertension, assessing the efficacy of an anti-cardiovascular disease (or hypertension) treatment in a subject using an ACE2 activator, monitoring the progress of a subject being treated with an ACE2 activator, selecting a subject with or susceptible to cardiovascular disease or an associated condition (such as stroke or heart disease), or hypertension, or acute lung injury, and/or treating a subject suffering from or susceptible to cardiovascular disease or an associated condition (such as stroke or heart disease), or hypertension. In certain embodiments, the invention provides: a kit for treating cardiovascular disease or an associated condition (such as stroke or heart disease), or hypertension, in a subject, the kit comprising diminazene or a pharmaceutically acceptable salt thereof, or prodrugs thereof, and instructions for use; in certain embodiments, the compound is diminazene aceturate.

In another aspect, the invention provides the use of a compound of the invention for the manufacture of a medicament for the treatment of cardiovascular disease or cardiopulmonary disease (including systemic or pulmonary hypertension) or cardiac or renal fibrosis.

The present methods can be performed on cells in culture, e.g. in vitro or ex vivo, or on cells present in an animal subject, e.g., in vivo. Compounds of the inventions can be initially tested in vitro using primary cultures of cells.

The present methods can be performed on cells in culture, e.g. in vitro or ex vivo, or on cells present in an animal subject, e.g., in vivo. Compound of the invention can be initially tested in vitro using cells from the respiratory tract from embryonic rodent pups (See e.g. U.S. Pat. No. 5,179,109-fetal rat tissue culture), or other mammalian (See e.g. U.S. Pat. No. 5,089,517-fetal mouse tissue culture) or non-mammalian animal models.

Alternatively, the effects of a compound of the invention can be characterized in vivo using animals models.

### 4. Pharmaceutical Compositions

The invention also provides a pharmaceutical composition, comprising an effective amount of diminazene or a pharmaceutically acceptable salt thereof and a pharmaceutically acceptable carrier. In a further embodiment, the effective amount is effective to treat cardiovascular or cardiopulmonary disease or an associated condition, including hypertension, or cardiac or renal fibrosis, as described previously.

In an embodiment, the compound of the invention is administered to the subject using a pharmaceutically-acceptable formulation, e.g., a pharmaceutically-acceptable formulation that provides sustained delivery of the compound of the invention to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration to a subject. In other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “pharmaceutically acceptable” refers to those compound of the inventions of the present invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier is “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing a compound of the invention(s) include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, more preferably from about 10 percent to about 30 percent.

Methods of preparing these compositions include the step of bringing into association a compound of the invention(s) with the carrier and, optionally, one or more accessory ingredients. In general, the formulations are prepared by uniformly and intimately bringing into association a compound of the invention with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the invention(s) as an active ingredient. A compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compound of the invention(s) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to the active compound of the invention(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more compound of the invention(s) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of a compound of the invention(s) include powders, sprays, ointments, pastes, creams, lotions, gels, solutions, patches and inhalants. The active compound of the invention(s) may be mixed under sterile conditions with a pharmaceutically-acceptable carrier, and with any preservatives, buffers, or propellants which may be required.

The ointments, pastes, creams and gels may contain, in addition to compound of the invention(s) of the present invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of the invention(s), excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The compound of the invention(s) can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a compound of the invention(s) to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Ophthalmic formulations, eye ointments, powders, solutions and the like, are also contemplated as being within the scope of the invention.

Pharmaceutical compositions of the invention suitable for parenteral administration comprise one or more compound of the invention(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of compound of the invention(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compound of the invention(s) are administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

Regardless of the route of administration selected, the compound of the invention(s), which may be used in a suitable hydrated form, and/or the pharmaceutical compositions of the present invention, are formulated into pharmaceutically-acceptable dosage forms by conventional methods known to those of skill in the art.

Actual dosage levels and time course of administration of the active ingredients in the pharmaceutical compositions of the invention may be varied so as to obtain an amount of the active ingredient which is effective to achieve the desired therapeutic response for a particular patient, composition, and mode of administration, without being toxic to the patient. An exemplary dose range is from 0.01 to 10 mg per day.

A preferred dose of the compound of the invention for the present invention is the maximum that a patient can tolerate and not develop serious or unacceptable side effects. In certain embodiments, the compound of the present invention is administered at a concentration of about 10 micrograms to about 100 mg per kilogram of body weight per day, about 0.1-about 10 mg/kg or about 1.0 mg-about 10 mg/kg of body weight per day. Ranges intermediate to the above-recited values are also intended to be part of the invention.

The invention is further illustrated by the following examples which should in no way should be construed as being further limiting.

### EXAMPLES

Emerging evidence suggests that ACE2 could be a new therapeutic target for cardiovascular disease (CVD) and hypertension. In this study, we used a molecular docking strategy combined with in vitro functional screening to search for novel ACE2 activators in support of this concept. Enzymatic assays with top-scoring compounds demonstrated that diminazene aceturate (DIZE), a known antiprotozoal drug used in humans, acts as a potent activator of ACE2. DIZE caused a dose-dependent increase in ACE2 activity and a maximal increase of 2.3 fold (Vmax) was observed with 50 μM. Intravenous administration of DIZE in Wistar Kyoto rats (WKYs) and spontaneously hypertensive rats (SHRs) caused a transient and dose-dependent decrease in mean arterial pressure (MAP). Although DIZE decreased MAP in both strains of rats, its efficacy was significantly higher in the SHRs; a dose of 5 mg/kg of DIZE triggered a MAP reduction of 50±9 mmHg in WKY rats (n=6) compared to 85±11 mmHg in SHRs (n=6). In addition to the strong effect in reducing blood pressure, the present study shows that DIZE does not significantly affect the heart rate during the acute IV bolus administration. These data suggest that DIZE may be useful for treatment of CVD and hypertension or serve as a lead compound for the discovery of next generation of drugs for the treatment of CVD and hypertension.

Accumulated evidence demonstrates that renin angiotensin system (RAS) plays a critical role in the control of blood pressure. RAS comprises two major axes: the first, represented by angiotensin converting enzyme (ACE)/angiotensin II (Ang II)/angiotensin type 1 receptor (AT1R) that is responsible for vasoconstrictive, proliferative, fibrotic and hypertrophic effects. The counteracting axis of the RAS is represented by angiotensin converting enzyme 2 (ACE2)/angl-7/Mas receptor (MasR) that plays a role in the vasoprotective actions.

Given the fact that crystal structure of ACE2 enzyme has been resolved, a structured based identification of ACE2 activators led to the discovery of xanthenone (XNT):

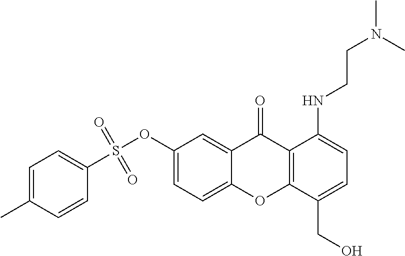

(1-[[2-(dimethylamino)ethyl]amino]-4-(hydroxymethyl)-7-[[4-methylphenylsulfonyl]oxy]-9H-xanthenone, XNT)

This approach has provided new insights as well as an important potential treatment of CVD and hypertension (HTN).

However, studies using XNT have brought to light key limitations of this lead compound which makes it less than ideal for preclinical trials. These limitations led us to undertake the structure based virtual screening approach for compounds that are in the FDA database in order to discover another compound that will circumvent the inherent limitations of XNT. This led to the identification of diminazene aceturate (DIZE). Presented herein is evidence that DIZE, an anti-protozoal agent is an ACE2 activator and causes decrease in blood pressure.

### Material and Methods

### Enzymes, Substrates, and Small Molecule Compounds

Recombinant ACE and ACE2 are obtained in purified form from R&D Systems, Minneapolis, Minn. (catalog ID: 929-ZN-10 and 933-ZN-10, respectively). Substrates for ACE (fluorogenic peptide V, Mca-RPPGFSAFK(Dnp)-OH, catalog ID: ES005), and for ACE2 (fluorogenic peptide VI, Mca-YVADAPK(Dnp)-OH, catalog ID: ES007) are obtained from R&D systems. Dry compounds are resuspended in 100% DMSO to prepare 100 mM stock solutions, according to the amount of compound and its molecular weight. Gentle heating to 60-80° C. is carried out to assist their solubilization. Compounds are further diluted to 50 mM stocks if clearly difficult to dissolve.

### Activity Assays

Activity of ACE and ACE2 are measured with a Spectra Max Gemini EM Florescence Reader (Molecular Devices). The enzyme removes the c-terminal dinitrophenyl moiety that quenches the inherent fluorescence of its 7-methoxycoumain group, resulting in an increase in fluorescence in the presence of enzyme activity. Fluorescence is measured with excitation and emission spectra of 328 nm and 392 nm, respectively. Reaction mixtures are prepared in 100 μl volumes and different concentrations of compound are tested against 10 μM substrate. 10 nM enzyme in 100 mM NaCl, 75 mM Tris, 0.5 μM ZnCl₂, at pH 7.4. Samples are read every 15-20 seconds for at least 30 minutes immediately after the addition of fluorogenic peptide substrate at 37° C. Assays, including controls, are performed in the presence of 1% dimethyl sulfoxide (DMSO). Although higher concentrations of NaCl increase the activity of ACE2 and ACE (Vickers et al. 2002), a low concentration of salt (100 mM NaCl) is used in the assays to allow for enhancement of enzymatic activity to be detectable. That is, using 1 M NaCl which gives a maximal enhancing effect from the Cl ions might not allow the compounds to further enhance the activity of the enzyme.

### Example 1

### Virtual Screening and Measurement of ACE2 Activity In Vitro

In silico screening includes docking of compounds into the crystal structure of the open conformation of ACE2 (PDBID 1R42) in 100 different orientations using the structural stringencies of the allosteric site. The top scoring compounds with the highest overall energy scores were tested under saturating substrate conditions for their ability to modulate human recombinant ACE2 enzyme (R&D Systems) activity in vitro. For a description of virtual screening methods, see also U.S. patent application Ser. No. 12/516,176, the contents of which are incorporated herein in their entirety.

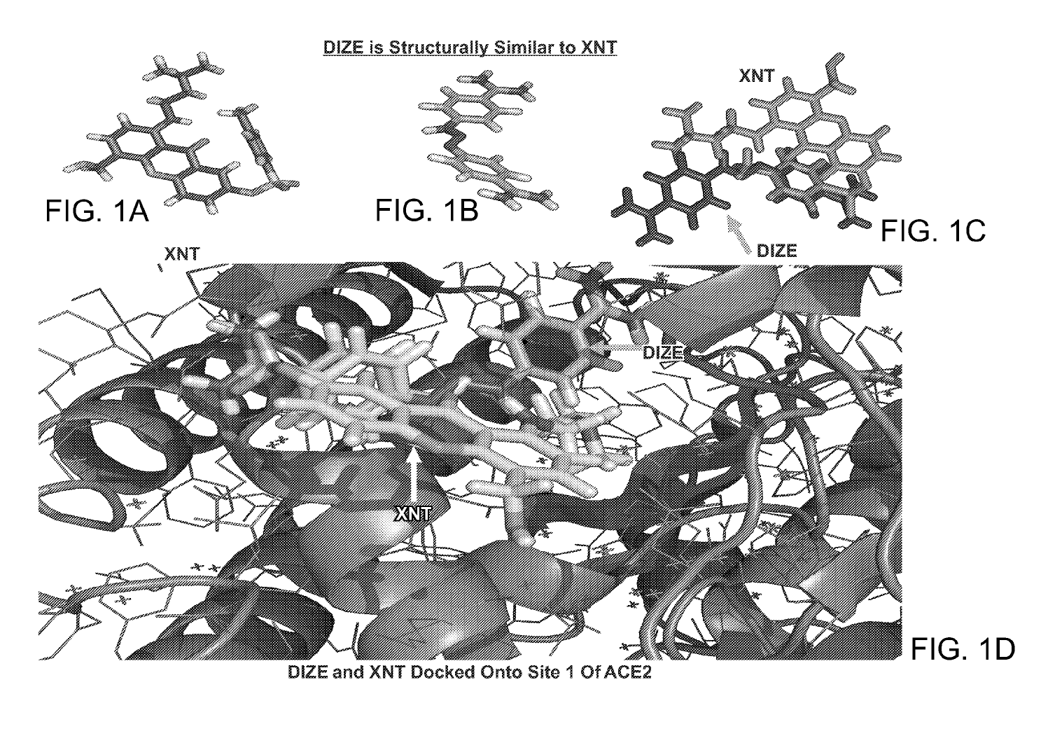

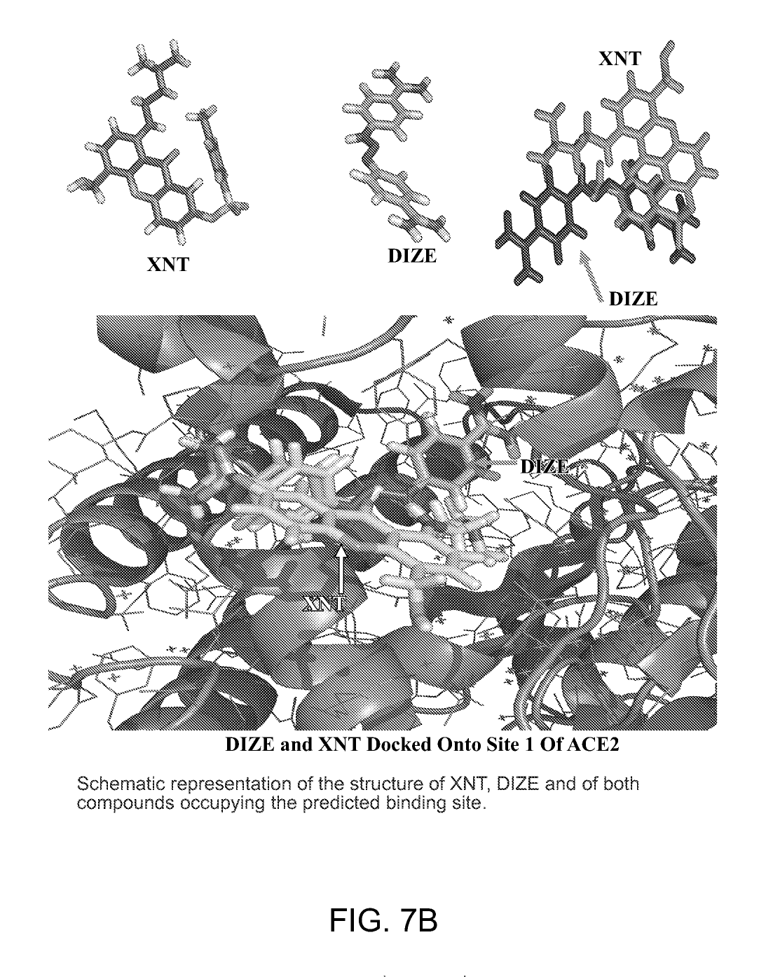

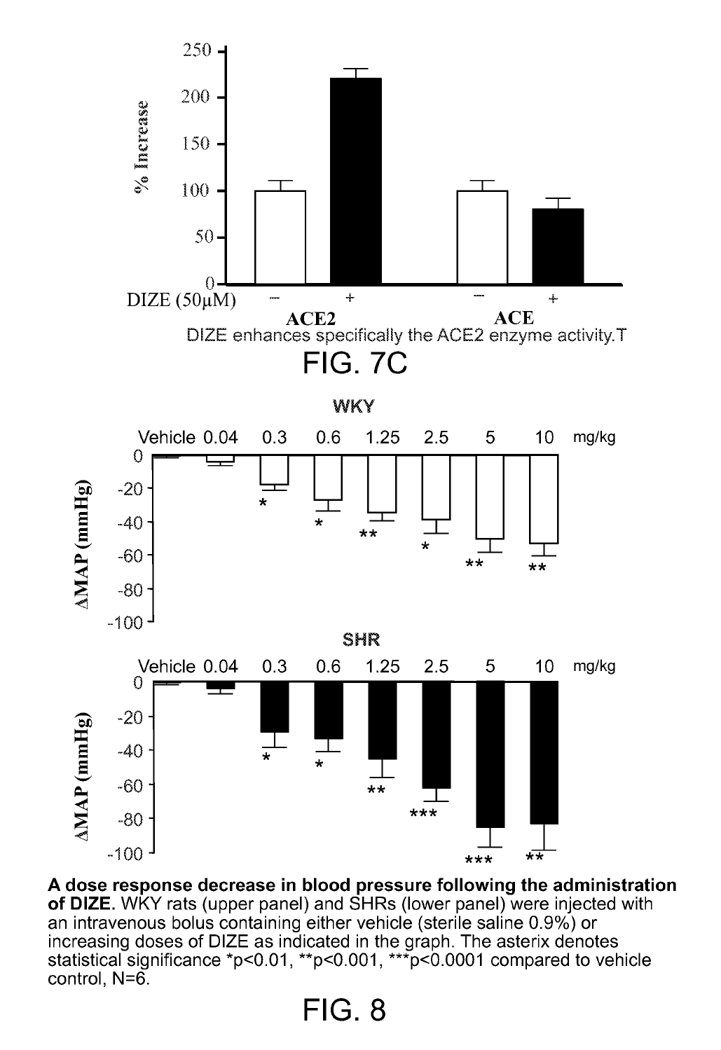

Molecular docking of DIZE to ACE2 is shown in FIGS. 1 and 7.

### Example 2

### Animals

Male Wistar-Kyoto (WKY) rats and spontaneously hypertensive rats (SHRs) were purchased from Charles River Laboratories (Wilmington, Mass., USA). All procedures were performed in conformity with approved Institutional Animal Care and Use Committee protocols and University of Florida regulations.

Direct Blood Pressure Measurements

Cannulation of the animals (WKY rats and SHRs) was performed 24 h prior to the start of the drug administration and direct blood pressure (BP) measurement. Animals were anesthetized with a mixture of ketamine, xylazine, and acepromazine (30, 6, and 1 mg/kg, respectively). The two types of the cannulae used were: 1) a polyethylene cannula (PE-50, Clay Adams) introduced into the carotid artery for direct BP measurements and 2) a silicone elastomer cannula (Helix Medical) introduced into the descending jugular vein for acute intravenous (IV) bolus injections of the drug. Both cannulae were filled with heparin saline (40 U/mL, Sigma), and sealed with stylets. The IV drug administration of diminazene aceturate (DIZE) (LKT laboratories, St. Paul, Minn., USA) and the dose-response curves in WKYs and SHRs were obtained in awake, freely moving animals after a 24 h recovery period. Doses of diminazene aceturate (DIZE) (0.04, 03, 0.6, 1.25, 2.5, 5 and 10 mg/kg) were administered as an IV bolus via the jugular cannula, and BP data recording was interfaced by a PowerLab (ADlnstruments) signal transduction unit. The data analysis was done using the Chart program (PowerLab system).

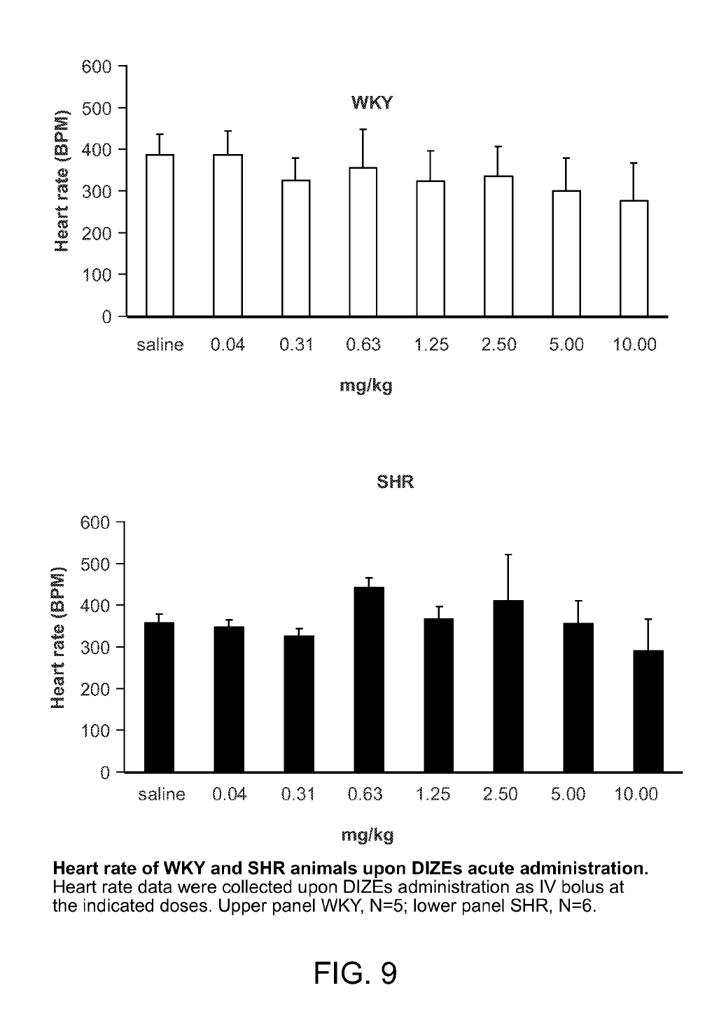

The results are shown in FIGS. 8 and 9.

### Example 3

We have investigated two models of pulmonary hypertension and one model of cardiac disease, myocardial infarction, In each of these cases pretreatment with DIZE completely prevents the development of these disorders. In pulmonary hypertension, the pulmonary pressure is nearly normalized and fibrosis is virtually absent. When DIZE is administered after the development of pulmonary hypertension, any further the progression of any further pathophysiology is arrested. In the heart model, pretreatment reduces infarct area and significantly improves cardiac function.

Another important finding is the effect of DIZE on stem cell function. Endothelial progenitor cells harvested from pulmonary hypertensive patients are dysfunctional and have decreased ability to repair vascular damage induced by the disease. DIZE treatment significantly improves this dysfunction.

### CONCLUSIONS

A structured-based approach combined with enzymatic assays in vitro, revealed diminazene aceturate (DIZE) as an ACE2 activator.

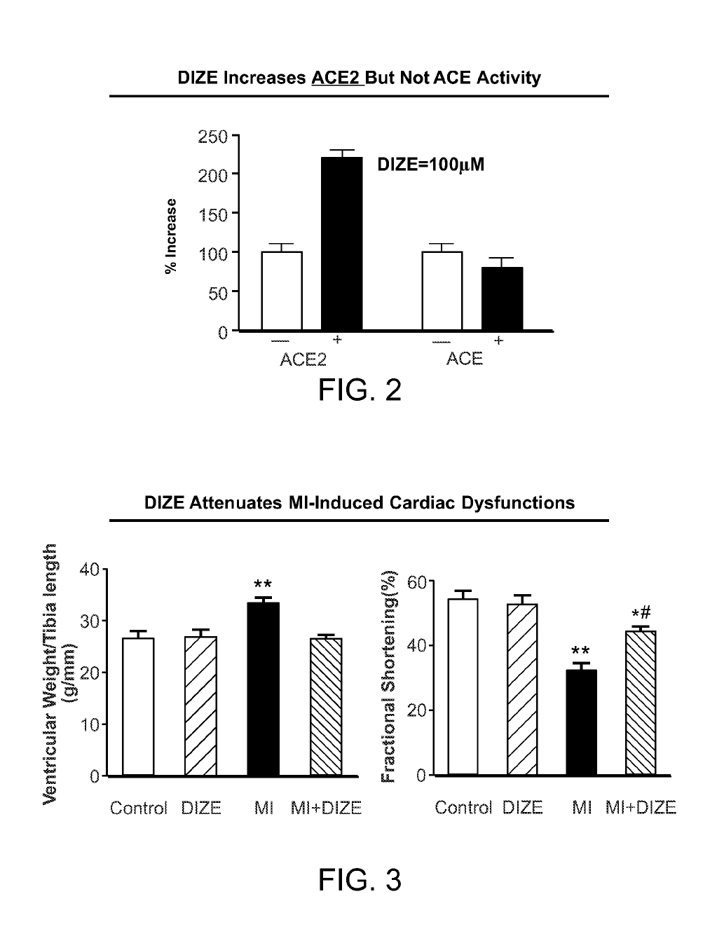

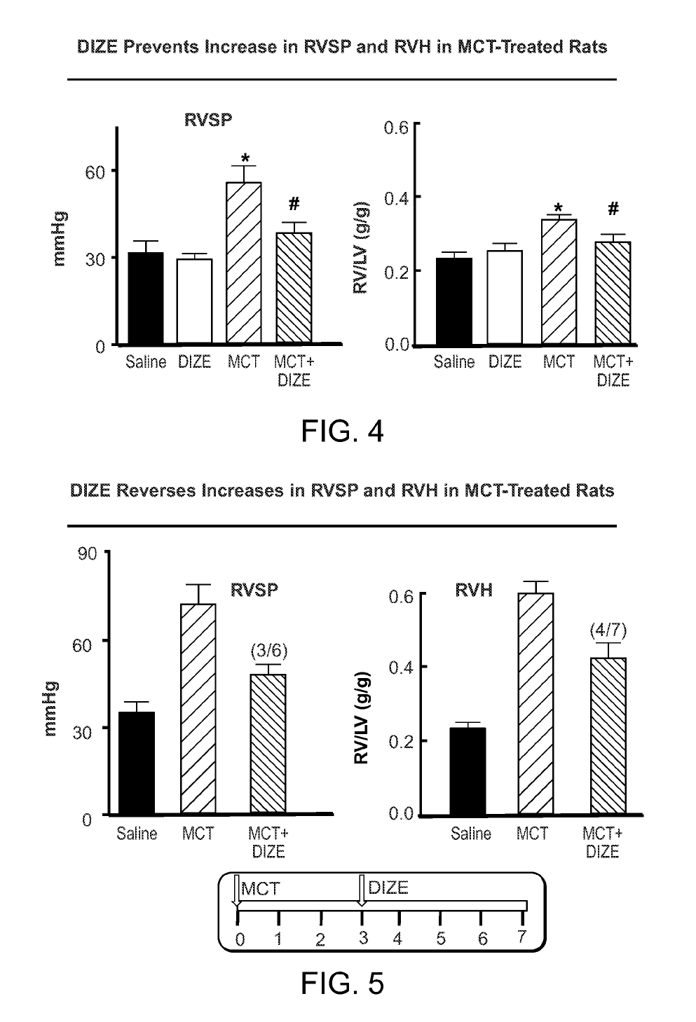

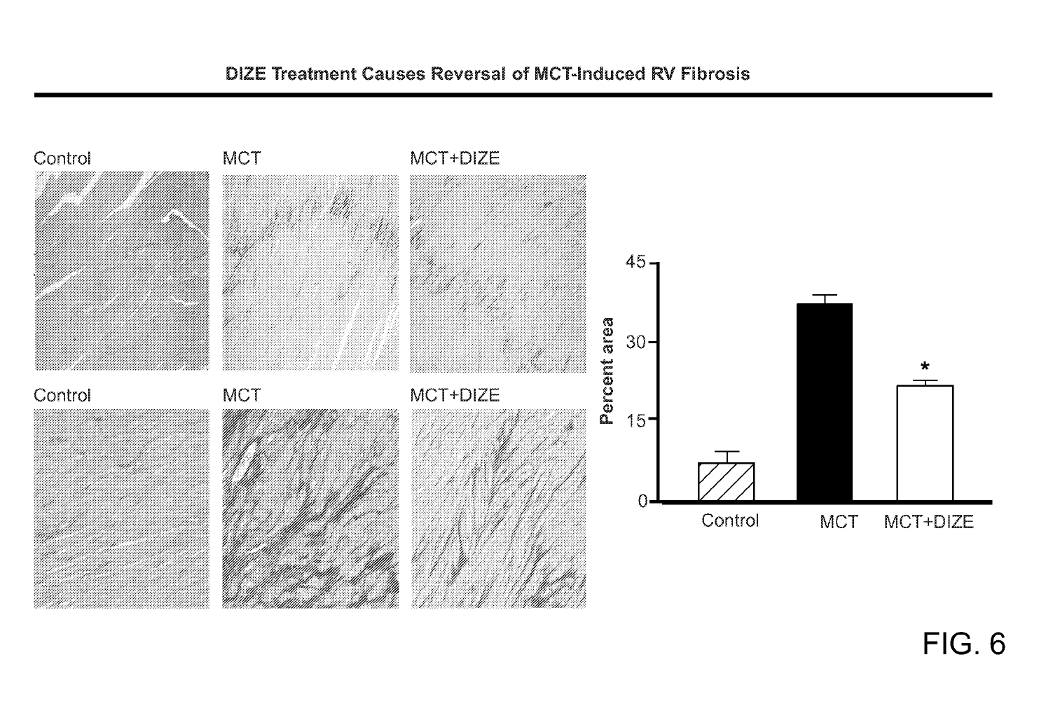

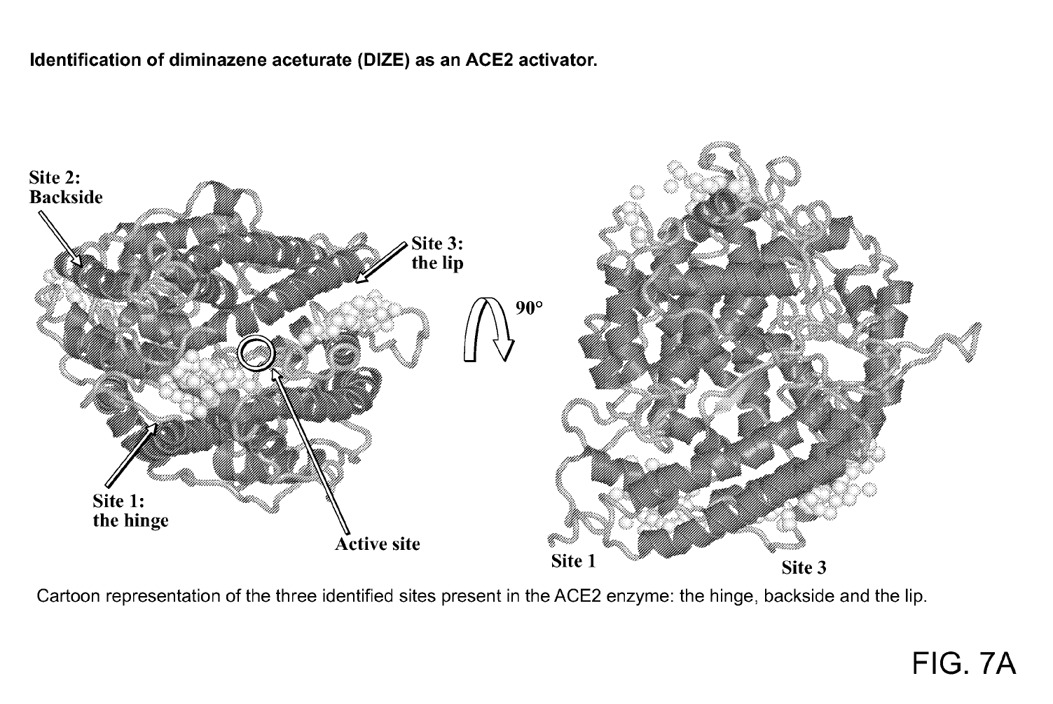

DIZE caused a dose-dependent increase in ACE2 activity and a maximal increase of 2.3 fold (Vmax) was observed with 50 μM. As shown in FIG. 2, increases ACE2 but not ACE activity.

Intravenous administration of DIZE triggers a transient and dose dependent decrease in mean arterial pressure (MAP) both in WKYs and SHRs.

DIZEs IV administration provokes a greater reduction in blood pressure in SHRs compared to WKY control rats, demonstrating a higher sensitivity of the hypertensive animals (SHRs).

DIZEs IV administration does not affect the heart rate of WKY and SHR animals

Collectively, the present work validates the activation of the ACE2 enzyme as an effective strategy in lowering blood pressure and confirms structured based identification of ACE2 activators as a good strategy in finding these compounds.

### REFERENCES

Asensio J. L., Canada F. J., Bruix M., Rodriguez-Romero A. and Jimenez-Barbero J. (1995) The Interaction of Hevein with N-Acetylglucosamine-Containing Oligosaccharides. Solution Structure of Hevein Complexed to Chitobiose. Eur. J. Biochem. 230, 621-633.
Ash E. L., Sudmeier J. L., Day R. M., Vincent M., Torchilin E. V., Haddad K. C., Bradshaw E. M., Sanford D. G. and Bachovchin W. W. (2000) Unusual ¹H NMR chemical shifts support (His) C(epsilon) 1 . . . O═C H-bond: proposal for reaction-driven ring flip mechanism in serine protease catalysis. PNAS 97, 10371-10376.
Atkinson M. A. and Leiter E. H. (1999) The NOD Mouse Model of Type 1 Diabetes: As Good as it Gets? Nat. Medicine 5, 601-604.
Babu M. M. (2003) NCl: A Server to Identify Non-Canonical Interactions in Protein Structures. Nucleic Acids Res. 31, 3345-3348.
Bader M. (2002) Role of the Local Renin-Angiotensin System in Cardiac Damage: A Minireview Focussing on Transgenic Animal Models. J. Mol. Cell. Cardiol. 34, 1455-1462.
Bajorath J. (2002) Integration of Virtual and High-Throughput Screening. Nat. Rev. Drug Discov. 1, 882-894.
Barclay A. N. (2003) Membrane Proteins with Immunoglobulin-Like Domains—A Master Superfamily of Interaction Molecules. Semin. Immunol. 15, 215-223.
Barton G. J. (1993) ALSCRIPT: A Tool to Format Multiple Sequence Alignments. Protein Eng. 6, 37-40.
Becker O. M., Marantz Y., Shacham S., Inbal B., Heifetz A., Kalid O., Bar-Haim S., Warshaviak D., Fichman M. and Noiman S. (2004) G Protein-Coupled Receptors: In Silico Drug Discovery in 3D. PNAS101, 11304-11309.
Berman H. M., Westbrook J., Feng Z., Gilliland G., Bhat T. N., Weissig H., Shindyalov I. N. and Bourne P. E. (2000) The Protein Data Bank. Nucleic Acids Res. 28, 235-242.
Binkowski T. A., Naghibzadeh S, and Liang J. (2003) CASTp: Computed Atlas of Surface Topography of Proteins. Nucleic Acids Res. 31, 3352-3355.
Bohacek R. S., McMartin C. and Guida W. C. (1996) The Art and Practice of Structure-Based Drug Design: A Molecular Modeling Perspective. Med. Res. Rev. 16, 3-50.
Borghi C., Bacchelli S., Degli Esposti D. and Ambrosioni E. (2004) A Review of the Angiotensin-Converting Enzyme Inhibitor, Zofenopril, in the Treatment of Cardiovascular Diseases. Expert Opin. Pharmacother. 5, 1965-1977.
Brunger A. T. (1992) Free R Value: A Novel Statistical Quantity for Assessing the Accuracy of Crystal Structures. Nature 355, 472-475.
Brunger A. T., Adams P. D., Clore G. M., DeLano W. L., Gros P., Grosse-Kunstleve R. W., Jiang J. S., Kuszewski J., Nilges M., Pannu N. S., Read R. J., Rice L. M., Simonson T. and Warren G. L. (1998) Crystallography & NMR System: A New Software Suite for Macromolecular Structure Determination. Acta Cryst. D54 (Pt 5), 905-921.
Burrell L. M., Johnston C. I., Tikellis C. and Cooper M. E. (2004) ACE2, A New Regulator of the Renin-Angiotensin System. Trends Endocrinol. Metab. 15, 166-169.
Cantoni C., Ponassi M., Biassoni R., Conte R., Spallarossa A., Moretta A., Moretta L., Bolognesi M. and Bordo D. (2003) The Three-Dimensional Structure of the Human NK Cell Receptor NKp44, a Triggering Partner in Natural Cytotoxicity. Structure 11, 725-734.

Card G. L., Blasdel L., England B. P., Zhang C., Suzuki Y., Gillette S., Fong D., Ibrahim P. N., Artis D. R., Bollag G., Milburn M. V., Kim S. H., Schlessinger J. and Zhang K. Y. (2005) Family of Phosphodiesterase Inhibitors Discovered by Cocrystallography and Scaffold-based Drug Design. Nat. Biotechnol. 23, 201-207.
Carter P., Andersen C. A. and Rost B. (2003) DSSPcont: Continuous Secondary Structure Assignments for Proteins. Nucleic Acids Res. 31, 3293-3295.
Chen B., Piletsky S, and Turner A. P. (2002) High Molecular Recognition: Design of “Keys”. Comb. Chem. High Throughput Screen. 5, 409-427.
Cleland W. W. (2000) Low-Barrier Hydrogen Bonds and Enzymatic Catalysis. Arch. Biochem. Biophys. 382, 1-5.
Collaborative Computational Project N. (1994) The CCP4 Suite: Programs for Protein Crystallography. Acta Cryst. D50, 760-763.
Colman P. M. (1988) Structure of Antibody-Antigen Complexes: Implications for Immune Recognition. Adv. Immunol. 43, 99-132.
Connolly M. L. (1983). Solvent-Accessible Surfaces of Proteins and Nucleic Acids. Science 221, 709-713.
Crackower M. A., Sarao R., Oudit G. Y., Yagil C., Kozieradzki I., Scanga S. E., Oliveira-dos-Santos A. J., da Costa J., Zhang L., Pei Y., Scholey J., Ferrario C. M., Manoukian A. S., Chappell M. C., Backx P. H., Yagil Y. and Penninger J. M. (2002) Angiotensin-Converting Enzyme 2 is an Essential Regulator of Heart Function. Nature 417, 822-828.
Dauter Z., Lamzin V. S, and Wilson K. S. (1997) The Benefits of Atomic Resolution. Curr. Opin. Struct. Biol. 7, 681-688.
DeLano W. L. (2002) The PyMOL Molecular Graphics System. DeLano Scientific, San Carlos, Calif., USA.
Derewenda Z. S., Derewenda U. and Kobos P. M. (1994) (His)C Epsilon-H . . . o═C<Hydrogen Bond in the Active Sites of Serine Hydrolases. J. Mol. Biol. 241, 83-93.
Derewenda Z. S., Lee L. and Derewenda U. (1995) The Occurrence of C—H . . . O Hydrogen Bonds in Proteins. J. Mol. Biol. 252, 248-262.
Desiraju G. R. (1996) The CH—O Hydrogen Bond: Structural Implications and Supramolecular Design. Acc. Chem. Res. 29, 441-449.
Desmyter A., Transue T. R., Ghahroudi M. A., Thi M. H., Poortmans F., Hamers R., Muyldermans S, and Wyns L. (1996) Crystal Structure of a Camel Single-Domain VH Antibody Fragment in Complex with Lysozyme. Nat. Struct. Biol. 3, 803-811.
Dobson C. M. (2004) Chemical Space and Biology. Nat. Insight. Intro. pp. 2
Donoghue M., Hsieh F., Baronas E., Godabout K., Gosselin M., Stagliano N., Donovan M., Woolf B., Robison K., Jeyaseelan R., Breitbart R. E. and Acton S. (2000) A Novel Angiotensin-Converting Enzyme-Related Carboxypeptidase (ACE2) Converts Angiotensin Ito Angiotensin 1-9. Cir. Res. 87, E1-E9.
Dyson H. J. and Wright P. E. (2005) Intrinsically Unstructured Proteins and Their Functions. Nat. Rev. Mol. Cell. Biol. 6, 197-208.
Emsley P. and Cowtan K. (2004) Coot: Model-Building Tools for Molecular Graphics. Acta Cryst. D60, 2126-2132.
Evans S. V. (1993) SETOR: Hardware-Lighted Three-Dimensional Solid Model Representations of Macromolecules. J. Mol. Graphics. 11, 134-138, 127-128.
Ewing T. J., Makino S., Skillman A. G. and Kuntz I. D. (2001) DOCK 4.0: Search Strategies for Automated Molecular Docking of Flexible Molecule Databases. J. Comput. Aided Mol. Des. 15, 411-428.
Guex N., Diemand A. and Peitsch M. C. (1999) Protein Modelling for All. Trends Biochem. Sci. 24, 364-367.
Guex N. and Peitsch M. C. (1997) SWISS-MODEL and the Swiss-PdbViewer: An Environment for Comparative Protein Modeling. Electrophoresis 18, 2714-2723.
Hendrickson W. A., Horton J. R. and LeMaster D. M. (1990) Selenomethionyl Proteins Produced for Analysis by Multiwavelength λnomalous Diffraction (MAD): A Vehicle for Direct Determination of Three-Dimensional Structure. Embo Journal 9, 1665-1672.
Hernandez Prada J. A., Haire R. N., Cannon J. P., Litman G. W. and Ostrov D. A. (2004) Crystallization and Preliminary X-Ray Analysis of VCBP3 from Branchiostoma Floridae. Acta Cryst. D60, 2022-2024.
Hiom K., Melek M. and Gellert M. (1998) DNA Transposition by the RAG1 and RAG2 Proteins: A Possible Source of Oncogenic Translocations. Cell 94, 463-470.
Hogue C. W. (1997) Cn3D: A New Generation of Three-Dimensional Molecular Structure Viewer. Trends Biochem. Sci. 22, 314-316.
Huentelman M. J., Zubcevic J., Katovich M. J. and Raizada M. K. (2004) Cloning and Characterization of a Secreted Form of Angiotensin-Converting Enzyme 2. Regul. Pept. 122, 61-67.
Irwin J. J. and Shoichet B. K. (2005) ZINC—A Free Database of Commercially Available Compounds for Virtual Screening. J. Chem. Inf. Model. 45, 177-182.
Jaffe E. K. (2005) Morpheeins—A New Structural Paradigm for Allosteric Regulation. Trends Biochem. Sci. 30, 490-497.
Jancarik J. and Kim S. H. (1991) Sparse Matrix Sampling: A Screening Method for Crystallization of Proteins. J. Appl. Cryst. 24, 409-411.
Jones T. A., Zou J. Y., Cowan S. W. and Kjeldgaard (1991) Improved Methods for Building Protein Models in Electron Density Maps and the Location of Errors in these Models. Acta Cryst. A47 (Pt 2), 110-119.
Kang B. S., Devedjiev Y., Derewenda U. and Derewenda Z. S. (2004) The PDZ2 Domain of Syntenin at Ultra-High Resolution: Bridging the Gap between Macromolecular and Small Molecule Crystallography. J. Mol. Biol. 338, 483-493.
Kantardjieff K. A. and Rupp B. (2003) Matthews Coefficient Probabilities: Improved Estimates for Unit Cell Contents of Proteins, DNA, and Protein-Nucleic Acid Complex Crystals. Protein Sci. 12, 1865-1871.
Katovich M. J., Grobe J. L., Huentelman M. and Raizada M. K. (2005) Angiotensin-Converting Enzyme 2 as a Novel Target for Gene Therapy for Hypertension. Exp. Physiol. 90, 299-305.
Kitchen D. B., Decornez H., Fun J. R. and Bajorath J. (2004) Docking and Scoring in Virtual Screening for Drug Discovery: Methods and Applications. Nat. Rev. Drug Discov. 3, 935-949.
Kolodny R., Koehl P. and Levitt M. (2005) Comprehensive Evaluation of Protein Structure Alignment Methods: Scoring by Geometric Measures. J. Mol. Biol. 346, 1173-1188.
Kuntz I. D., Blaney J. M., Oatley S. J., Langridge R. and Ferrin T. E. (1982) A Geometric Approach to Macromolecule-Ligand Interactions. J. Mol. Biol. 161, 269-288.
Langer T. and Hoffmann R. D. (2001) Virtual Screening: An Effective Tool for Lead Structure Discovery? Curr. Pharm. Des. 7, 509-527.
Laskowski R. A., Moss D. S, and Thornton J. M. (1993) Main-Chain Bond Lengths and Bond Angles in Protein Structures. J. Mol. Biol. 231, 1049-1067.
Leahy D. J., Axel R. and Hendrickson W. A. (1992) Crystal Structure of A Soluble Form of the Human T Cell Coreceptor CD8 at 2.6 A Resolution. Cell 68, 1145-1162.
Lipinski C. A., Lombardo F., Dominy B. W. and Feeney P. J. (1997) Experimental and Computational Approaches to Estimate Solubility and Permeability in Drug Discovery and Development Settings. Adv. Drug Delivery Rev. 23(1-3), 3-25.
Matthews B. W. (1968) Solvent Content of Protein Crystals . . . I Mol. Biol. 33, 491-497.
Matthews B. W. (1976) X-Ray Crystallographic Studies of Proteins. Ann. Rev. Phys. Chem. 27, 493-523.
McCammon J. A. (2005) Target Flexibility in Molecular Recognition. Biochim. Biophys. Acta. 1754, 221-4.
McDonald I. K. and Thornton J. M. (1994) Satisfying Hydrogen Bonding Potential in Proteins. J. Mol. Biol. 238, 777-793.
McPherson A. (1999) Crystallization of Biological Macromolecules (Cold Spring Harbor Laboratory, 1st edition) pp. 586.
McRee D. E. (1999) XtalView/Xfit—A Versatile Program for Manipulating Atomic Coordinates and Electron Density. J. Struct. Biology 125, 156-165.
Meng E. C., Gschwend D. A., Blaney J. M. and Kuntz I. D. (1993) Orientational Sampling and Rigid-Body Minimization in Molecular Docking. Proteins 17, 266-278.
Meng E. C., Shoichet K. and Kuntz I. D. (1992) Automated Docking with Grid Based Energy Evaluation. J. Comp. Chem. 13, 505-524.
Merritt E. A. (1994) Raster3D Version 2.0. A Program for Photorealistic Molecular Graphics. Acta Cryst. D50, 869-873.
Merritt E. A. (1999) Comparing Anisotropic Displacement Parameters in Protein Structures. Acta Cryst. D55 (Pt 12), 1997-2004.
Miura K., Daviglus M. L., Greenland P. and Stamler J. (2001) Making Prevention and Management of Hypertension Work. J. Hum. Hypertens. 15, 1-3.
Murasato Y., Hirakawa H., Harada Y., Nakamura T., Hayashida Y., 1998. Effects of systemic hypoxia on R—R interval and blood pressure variabilities in conscious rats. Am J Physiol 275, H797-H804.
Myers G. and Durbin R. (2003) A Table-Driven, Full-Sensitivity Similarity Search Algorithm. J. Comput. Biol. 10, 103-117.
Natesh R., Schwager S. L., Sturrock E. D. and Acharya K. R. (2003) Crystal Structure of the Human Angiotensin-Converting Enzyme-Lisinopril Complex. Nature 421, 551-554.
Otwinowski Z., Borek D., Majewski W. and Minor W. (2003) Multiparametric Scaling of Diffraction Intensities. Acta Crystallographica. Section a, Crystal Physics, Diffraction, Theoretical and General Crystallography 59, 228-234.
Otwinowski Z. and Minor W. (1997) Processing of X-ray Diffraction Data Collected in Oscillation Mode. Methods Enzymol. 276, 307-326.
Peitsch M. C. (1996) ProMod and Swiss-Model: Internet-Based Tools for Automated Comparative Protein Modelling. Biochem. Soc. Trans. 24, 274-279.
Peitsch M. C., Schwede T. and Guex N. (2000) Automated Protein Modelling—the Proteome in 3D. Pharmacogenomics 1, 257-266.
Porta A., Montano N., Furlan R., Cogliati C., Guzzetti S., Gnecchi-Ruscone T., Malliani A., Chang H. S., Staras K., Gilbey M. P., 2004. Automatic classification of interference patterns in driven event series: application to single sympathetic neuron discharge forced by mechanical ventilation. Biol Cybern 91, 258-273.
Ruiz-Ortega M., Lorenzo 0., Ruperez M., Estevan V., Suzuki Y., Mezzano S., Plaza J. J. and Egido J. (2001) Role of the Renin-Angiotensin System in Vascular Diseases: Expanding the Field. Hypertension. 38, 1382-1387.
Schneider T. R. and Sheldrick G. M. (2002) Substructure Solution with SHELXD. Acta Cryst. D58, 1772-1779.
Schwede T., Kopp J., Guex N. and Peitsch M. C. (2003) SWISS-MODEL: An Automated Protein Homology-Modeling Server. Nucleic Acids Res. (Online) 31, 3381-3385.
Sheldrick G. M. and Schneider T. R. (1997) SHELXL: High-Resolution Refinement. Methods Enzymol. 277, 319-343.
Soares P. P., da Nobrega A. C., Ushizima M. R., Irigoyen M. C., 2004. Cholinergic stimulation with pyridostigmine increases heart rate variability and baroreflex sensitivity in rats. Auton Neurosci. June 30; 113(1-2):24-31.
Streltsov V. A., Varghese J. N., Carmichael J. A., Irving R. A., Hudson P. J. and Nuttall S. D. (2004) Structural Evidence for Evolution of Shark Ig New Antigen Receptor Variable Domain Antibodies from a Cell-Surface Receptor. Proc. Natl. Acad. Sci. USA 101, 12444-12449.
Suri M. F. K., Kirmani J. F. and Divani A. A. AHA News Release September 3, 2004, at www.americanheart.org.
Taylor R. and O. Kennard (1982) Crystallographic Evidence for the Existence of CH—O, CH—N, and CH—Cl Hydrogen Bonds. J. Am. Chem. Soc. 104, 5063-5070.
Terwilliger T. C. (2000) Maximum-Likelihood Density Modification. Acta Cryst. D56, 965-972.
Terwilliger T. C. (2002) Automated Main-Chain Model Building by Template Matching and Iterative Fragment Extension. Acta Cryst. D59, 38-44.
Terwilliger T. C. and Berendzen J. (1999) Automated MAD and MIR Structure Solution. Acta Cryst. D55 (Pt 4), 849-861.
Tipnis S. R., Hooper N. M., Hyde R., Karran E., Christie E. and Turner A. J. (2000) A Human Homolog of Angiotensin-Converting Enzyme. Cloning and Functional Expression as a Captopril-Insensitive Carboxypeptidase. J. Biol. Chem. 275, 33238-33243.
Towler P., Staker B., Prasad S. G., Menon S., Tang J., Parsons T., Ryan D., Fisher M., Williams D., Dales N. A., Patane M. A. and Pantoliano M. W. (2004) ACE2 X-Ray Structures Reveal a Large Hinge-Bending Motion Important for Inhibitor Binding and Catalysis. J. Biol. Chem. 279, 17996-18007.
Unger T. (2003) Blood Pressure Lowering and Renin-Angiotensin System Blockade. J. Hypertens. 21 suppl 6, S3-S7.
van Gunsteren W. F. (1996) Biomolecular Simulation: The GROMOS96 Manual and User Guide. 4-31.
Vickers C., Hales P., Kaushik V., Dick L., Gavin J., Tang J., Godbout K., Parsons T., Baronas E., Hsieh F., Acton S., Patane M., Nichols A. and Tummino P. (2002) Hydrolysis of Biological Peptides by Human Angiotensin-Converting Enzyme-Related Carboxypeptidase. J. Biol. Chem. 277, 14838-14843.
Wahl M. C. and Sundaralingam M. (1997) C—H . . . O Hydrogen Bonding in Biology. Trends Biochem. Sci. 22, 97-102.
Waki H., Katahira K., Polson J. W., Kasparov S., Murphy D., Paton J. F., 2006. Automation of analysis of cardiovascular autonomic function from chronic measurements of arterial pressure in conscious rats. Exp Physiol. January; 91(1):201-13.
Wallace A. C., Laskowski R. A. and Thornton J. M. (1995) LIGPLOT: A Program to Generate Schematic Diagrams of Protein-Ligand Interactions. Protein Eng. 8, 127-134.
Walters W. P., Stahl M. T. and Murcko M. A. (1998) Virtual Screening—An Overview. Drug Discov. Today 3, 160-178.

The disclosures of each and every patent, patent application and publication cited herein are hereby incorporated herein by reference in their entirety.

Although the invention has been disclosed with reference to specific embodiments, it is apparent that other embodiments and variations of the invention may be devised by others skilled in the art without departing from the true spirit and scope of the invention. The claims are intended to be construed to include all such embodiments and equivalent variations.

1. A method of treating a subject suffering from or susceptible to cardiovascular disease, hypertension disease, acute lung injury, liver, cardiac, pulmonary, vascular or renal fibrosis, pulmonary hypertension, chronic obstructive pulmonary disease (COPD), heart failure, diabetes or a diabetes-associated condition, cardiac damage induced by anticancer drugs, cardiac damage from myocardial infarction, liver damage, or stroke, the method comprising administering to a subject in need thereof a therapeutically effective amount of diminazene or a pharmaceutically acceptable salt thereof, to thereby treat the subject.

2.-11. (canceled)

12. A method of treating or preventing progression of tissue damage or pathophysiology in a subject suffering from pulmonary hypertension, the method comprising administering to the subject suffering from pulmonary hypertension an effective amount of diminazene or a pharmaceutically acceptable salt thereof, such that the progression of tissue damage or pathophysiology is treated or prevented.

13. A method of improving cardiac function in a subject suffering from myocardial infarction, the method comprising administering to the subject an effective amount of diminazene or a pharmaceutically acceptable salt thereof, such that cardiac function in the subject is improved.

14. (canceled)

15. (canceled)

16. A method of improving function of endothelial progenitor cells in a pulmonary hypertensive subject, the method comprising administering to the subject an effective amount of diminazene or a pharmaceutically acceptable salt thereof, such that the function of endothelial progenitor cells in the subject is improved.

17. The method of claim 1, wherein the diminazene or a pharmaceutically acceptable salt thereof is diminazene aceturate.

18. (canceled)

19. (canceled)
